(19) United States
(12) Patent Application Publication
(10) Pub. No.: US 2009/0205106 A1
(43) Pub. Date: Aug. 20, 2009

(54) WRIST BAND

(76) Inventor: Dae Up Sohn, Seoul (KR)

Correspondence Address: IPLA P.A., 3580 WILSHIRE BLVD., 17TH FLOOR, LOS ANGELES, CA, 90010, US

(21) Appl. No.: 12/300,082

(22) PCT Filed: May 8, 2007
(86) PCT No.: PCT/KR2007/002249

§ 371 Date: Nov. 7, 2008

(30) Foreign Application Priority Data
May 8, 2006 (KR) 10-2006-0040964

(51) Int. Cl. A41D 20/00; A41D 27/20; A61F 7/00; A61F 7/10

(52) U.S. Cl. 2/170; 2/247; 607/111; 607/112; 607/114

(57) ABSTRACT

Disclosed is a wrist band. The wrist band comprises a cylinder-shaped band body having a plurality of water absorbing pockets receiving a water absorbing agent therein and surrounding a user's wrist, one or more elastic bands for elastically connecting the pair of water absorbing agent pockets with each other, and a water absorbing agent chemical received in the band body. The wrist band performs a physical function of tightening to thereby pressurize a user's wrist when a user wears it, and can enhance an exercise effect. In addition, the wrist band can adjust body temperature and provide a fomentation effect thanks to the use of a water absorbing agent. Furthermore, the wrist band can easily absorb sweat from a body, so that it can be used like a towel.

### TECHNICAL FIELD

The present invention relates to a wrist band, and more particularly to a wrist band which is capable of absorbing sweat, adjusting the temperature of a user's body by holding water at a proper temperature and enhancing the exercise effect by increasing the weight of the user's body due to the presence of the water.

### BACKGROUND ART

A wrist band is generally worn by a user when the user takes exercise in a way that extensively uses the wrist, or in order to fix or protect the user's wrist when the user has injured his or her wrist.

Therefore, known wrist bands generally comprise a band body having a uniform width and surrounding a user's wrist and a pair of Velcro tapes attached to respective ends of the band body.

The known wrist band is worn through a method such that a user first applies and then presses one end (a first end) of the band body against a joint of the user's wrist, then pulls the other end (a second end) of the band body so that the band body is completely wrapped around the wrist, and finally overlaps respective ends of the wrist band so that the wrist band is held on the user's wrist by the pair of Velcro tapes provided on respective ends of the band body.

As described above, the known wrist band is worn in a manner such that the second end is pulled by a user so that the Velcro tapes provided at the first end and the second end overlap while the user continues to press the band body against the user's wrist after placing the band body on a joint of the user's wrist. Accordingly, there is difficulty in using the known wrist band because a user has to put on the wrist band using only one hand.

In addition, general known wrist bands have been used for the simple purpose of applying a pressure to the area of a user's wrist or partially absorbing external shocks when a user wears the wrist band.

Meanwhile, in recent years, interest in health and well-being has increased. As a result, interest in exercise, such as aerobic exercise, to strengthen human bodies has also increased.

In particular, of the aerobic exercises, the use of weights such as dumbbells or sand bags in a form in which a user wears the weight around his or her wrist, is increasing because the use of the weight enhances the increase in the muscular strength of arms and the exercise effect.

However, when doing aerobic exercises using dumbbells, the range of exercise is limited because a user must do exercise while holding the dumbbells. According to these circumstances, such exercises lead to injury or side effects such as calluses in the palm.

In addition, in the case of doing exercise wearing a sand bag, there is a problem in that sweat excreted from a user's body during exercise is not properly absorbed to the sand bag. Accordingly, a user has an unpleasant feeling due to the skin discomfort. The wrist band of the present invention can not only eliminate such side effects but also serves as a towel.

### DISCLOSURE OF INVENTION

### Technical Problem

Accordingly, the present invention has been devised in consideration of the aforementioned problems and conditions, and it is a feature of the present invention to provide a wrist band which is capable of adjusting the temperature of a user's body, enhancing exercise effect by holding water therein so as to serve as a weight and performing a wrist protection function.

A water absorbing agent embedded in the wrist band according to the invention can absorb a large amount of water. The water absorbing agent has a very small volume when dry but expands to have quite a large volume when it absorbs water.

The wrist band according to the invention will be useful in the event that the temperature of a human body must be decreased, or when a user wants to keep his or her body cool. If a user wears the wrist band around his or her wrist in a state in which the water absorbing agent in the write band holds a sufficient amount of cool water having a temperature lower than the body temperature of the user, heat is transferred from the user's body to the wrist band. This accounts for the body temperature adjusting effect.

Such a body temperature adjusting effect is a remarkable advantage from a physical aspect, that is, a temperature transmission aspect, in comparison with known wrist bands. In the case in which a known temperature control means such as a plastic bag sealed in a state in which water is held therein is used to adjust the body temperature, the known temperature-adjusting means provides only a function of adjusting temperature by means of heat transmission. However, according to the invention, the wrist band can effectively adjust body temperature by the use of both of heat transmission and evaporation.

That is, the present invention confers a great advantage in that the wrist band uses the theory of energy transfer due to evaporation based on the physical phenomenon in which moisture remaining in the water absorbing agent evaporates while taking heat away from a human body. In addition, since the wrist band operates based on the temperature difference between the water and the human body, the body cooling effect can last for a long period because it continuously takes heat from a human body until it has completely dried.

Further, other known body temperature adjusting products have been inconvenient for a user to use or wear them. However, since the body temperature adjusting product (wrist band) according to the invention is easily worn around a user's wrist, its advantage, in which it is convenient to use, is conspicuous.

The wrist band according to the invention can not only adjust the body temperature but also enhance the exercise effect due to its weight. The wrist band according to the invention can be easily used for the purpose of strengthening muscles by virtue of its weight, and it can also be used for the purpose of performing a physical function of tightening and pressing the user's wrist when it is worn by a user.

The water absorbing agent incorporated in the wrist band according to the invention can absorb a great amount of water. Accordingly, it can act to absorb sweat. That is, the wrist band can absorb not only sweat coming out from the user's wrist during exercise but also can easily eliminate sweat from a user's face when the user wipes his or her face with the wrist band. That is, the wrist band can be used like a towel during exercise.

### Technical Solution

In order to solve the various problems encountered in the known art, and in order to achieve the above described advantageous effects and features, in accordance with the present invention, there is provided a wrist band comprising a band body having a cylinder shape and having a plurality of water absorbing agent pockets for receiving a water absorbing agent therein, in which the water absorbing pockets are formed by seaming the band body in a longitudinal direction thereof, the water absorbing agent received in the band body, and one or more elastic bands are provided to add elasticity to the band body.

The water absorbing agent may be acrylate copolymer.

The band body may be made of a cloth which is porous and can absorb sweat.

The plurality of water absorbing agent pockets may be formed by dividing the band body by seaming it. That is, it is desired that the water absorbing agent be provided in the form of water absorbing agent packs so that the water absorbing agent is received in the water absorbing agent pockets, defined by seams.

The seams may be formed in the longitudinal direction of the arm. That is, by dividing the band body by providing seams in the longitudinal direction of the arm, it is possible to prevent the water absorbing agent from gathering together at one position. The elastic band may be made of cloth having elasticity or of rubber.

The water absorbing agent pocket, receiving the water absorbing agent therein, can be formed by dividing the band body by seaming it in the longitudinal direction of the arm, or in a direction orthogonal to the longitudinal direction.

According to another aspect of the present invention, the seams that divide the band body into the water absorbing agent pockets can extend in both the longitudinal direction and the direction orthogonal to the longitudinal direction.

According to a further aspect of the present invention, the seams can be formed in any direction as long as they can enable the water absorbing agent to be received in the band body in a well-distributed form in addition to the longitudinal direction and the direction orthogonal to the longitudinal direction.

### Advantageous Effects

According to the present invention, it is possible to absorb sweat, adjust the body temperature of a user by making the wrist band hold water at a proper temperature in advance, and enhance the exercise effect by the application of its weight. That is, if a user wears the wrist band according to the invention, the wrist band can not only provide the physical effect such that it functions to tighten and pressurize a user's wrist, but also enhance the exercise effect. Furthermore, the wrist band can adjust the body temperature and also provide a fomenting effect by the water absorbing function of the water absorbing agent incorporated in the wrist band. Still further, the wrist band can be used like a hand towel during exercise because it easily absorbs sweat from a body. Yet further, the wrist band is realized in the form of a cylinder, and, due to its elasticity, the wrist band therefore has an advantage in that it can be easily put on by a user.

### BRIEF DESCRIPTION OF THE DRAWINGS

FIG. 1 is a perspective view illustrating a wrist band according to a first embodiment of the invention;

FIG. 2 is a cross-sectional view illustrating the wrist band shown in FIG. 1;

FIG. 3 is a view illustrating the state of the wrist band shown in FIG. 1 in use;

FIG. 4 is a perspective view illustrating a wrist band according to a second embodiment of the present invention;

FIG. 5 is a perspective view illustrating a wrist band according to a third embodiment of the present invention;

FIG. 6 is a perspective view illustrating a wrist band according to another embodiment of the present invention; and

FIG. 7 is a perspective view illustrating a wrist band according to a further embodiment of the present invention.

### BEST MODE FOR CARRYING OUT THE INVENTION

Hereinafter, embodiments of the present invention will be described in detail with reference to the accompanying drawings.

Before providing an explanation of the embodiments of the invention, it is to be noted that in the plurality of embodiments of the invention, elements having like configurations are denoted by like reference numerals and symbols. Such like elements are explained representatively in the description relating to the first embodiment of the invention. In the description relating to elements other than those of the first embodiment, only such elements will be denoted with different reference numerals and signs.

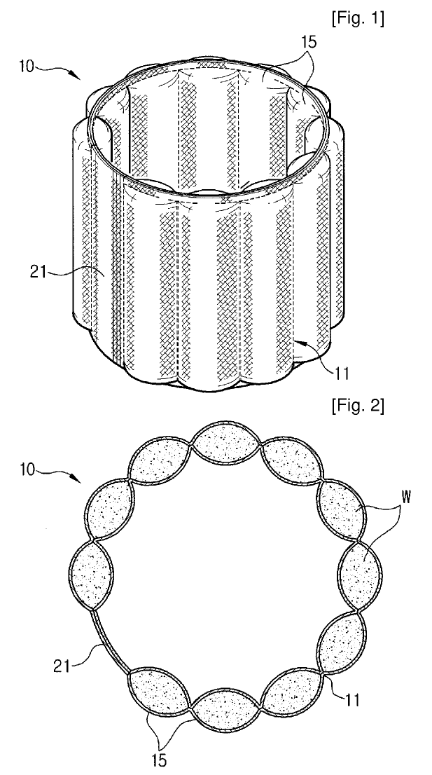

FIG. 1 is a perspective view illustrating a wrist band according to a first embodiment of the invention, and FIG. 2 is a cross-sectional view illustrating the wrist band shown in FIG. 1. As shown in FIG. 1 and FIG. 2, the wrist band 10 according to the first embodiment comprises a band body 11 that is worn around the wrist A. The band body 11 is in the form of a cylinder and has a uniform width.

The band body 11 is preferably made of a cloth which is porous and can absorb sweat.

The band body 11 has a plurality of water absorbing agent pockets 15 for receiving a water absorbing agent W therein. Seams are provided to the band body in the longitudinal direction of the arm A in order to divide the space of the band body into the water absorbing agent pockets. This configuration prevents the water absorbing agent W from gathering at one point.

The water absorbing agent W received in the water absorbing agent pockets 15 is preferably a chemical compound having high water absorbing efficiency.

The water absorbing agent W is preferably a composite in which acrylate copolymer and sodium salt are primary ingredients, or acrylate copolymer alone is a primary ingredient.

The aforementioned water absorbing agent exists in the form of a particle when it is in the dried state but in the form of a gel when it holds water. The water absorbing agent has high water absorbing efficiency and also has heat-retention efficiency. The water absorbing agent has a property in which its weight increases when it absorbs and holds water, but decreases when it is dry.

Such a water absorbing agent W is first received in the water absorbing agent pockets 15 in a manner in which it is capable of flowing, and is then sealed by seams. When it is sealed, the water absorbing agent 15 does not flow outside the water absorbing agent pockets 15.

On the other hand, the wrist band 10 according to the first embodiment includes an elastic band 21 which connects a pair of adjacent water absorbing agent pockets 15 to each other.

The elastic band 21 may be formed of cloth having elasticity or of rubber.

When applying the band body 11 with the elastic band 21 to the wrist A, the elastic band provides elasticity. Thanks to such elasticity, the elastic band can be deformed, and therefore the size of the wrist band can be varied. That is, the elastic band plays a role on making it easy for a user to put on the wrist band.

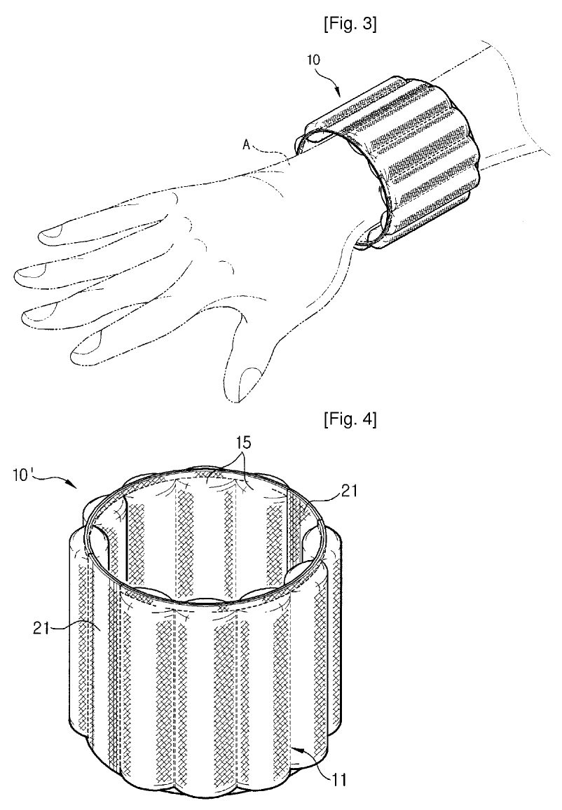

With reference to such configurations, the usage procedure of the wrist band according to the first embodiment will be described below. As shown in FIG. 3, a user passes his or her wrist through the center hole of the band body 11 so that the band body 11 surrounds the joint portion of the user's wrist A. At this time, the elastic band 21 provided between the pair of adjacent water absorbing agent pockets 15 is elastically deformed so that the wrist band 11 is worn around the joint portion of the user's wrist A. Through this simple method, the wrist band 10 can be easily put on by a user.

However, if the wrist band 10 is wet, the water absorbing agent W received in the water absorbing agent pockets 15 increases in weight as it absorbs water. In this state, if a user puts on the wet wrist band, the weight applied to the user's arm also increases.

In this instance, if the user does aerobic exercise while wearing the wet wrist band 10, the wrist band 10 tightens and pressurizes the user's wrist A. Thus, the wet wrist band can protect the user's wrist A during the use thereof, and can also enhance the exercise effect by applying its weight to the user's arm to strengthen the user's arm muscles.

In addition, the water absorbing agent W has the heat-retention function. Accordingly, the wrist band can perform the functions of adjusting the body temperature and providing a fomentation effect. Furthermore, since the water absorbing agent W can easily absorb sweat from the body, the wrist band can serve as a towel worn on the wrist during exercise.

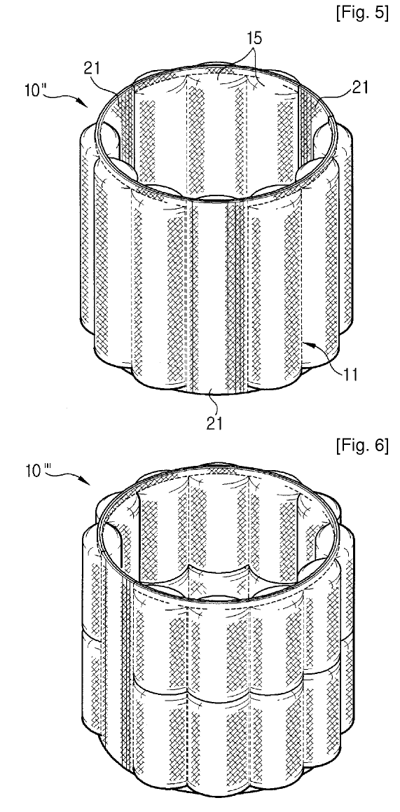

FIG. 4 illustrates a wrist band 10′ according to a second embodiment of the invention. As shown in this figure, the wrist band 10′ according to the second embodiment, unlike the first embodiment, is provided with a pair of elastic bands 21. Thanks to this, the wrist band 10′ according to the second embodiment can be more easily put on by a user than the wrist band 10 according to the first embodiment.

FIG. 5 illustrates a wrist band 10″ according to a third embodiment of the invention. The wrist band 10″ includes three elastic bands 21 arranged at regular intervals between the water absorbing agent pockets 15.

As described above, the wrist band according to the invention is formed into a cylinder form by the use of one or more elastic bands 21 elastically connecting the band body comprising the plurality of water absorbing agent pockets receiving the water absorbing agent therein. Thus, it is possible to easily and conveniently wear the wrist band according to the invention even using one hand.

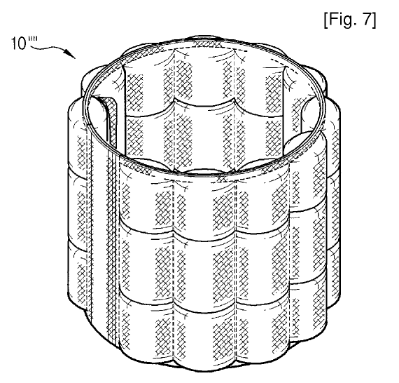

FIG. 6 and FIG. 7 are perspective views illustrating wrist bands according to other embodiments of the invention.

In the wrist band according to the invention, the water absorbing agent pockets for receiving the water absorbing agent therein are formed by dividing the band body using seams extending in the longitudinal direction of the arm. Alternatively, the water absorbing agent pockets for receiving the water absorbing agent therein can also be formed by seams extending in the direction orthogonal to the longitudinal direction of the arm.

Further, the seams can also extend both in the longitudinal direction of the wrist and in the direction orthogonal to the longitudinal direction. Yet further, according to the invention, the seams may extend in any direction, as long as they can hold the water absorbing agent in a well-distributed state.

The number of elastic bands, which is exemplified in the aforementioned embodiments, can be changed according to necessity.

It will be apparent to those skilled in the art that the present invention is not limited to the aforementioned embodiments, but that various modifications and variations to the embodiments may be made without departing from the spirit or scope of the invention. Accordingly, it is intended that the present invention cover the modifications and variations of this invention, provided they fall within the scope of the appended claims and their equivalents.

1. A wrist band, comprising:
a cylinder-shaped band body having a plurality of water absorbing pockets for receiving a water absorbing agent therein, in which the water absorbing pockets are divided and formed by seams;
a chemical compound serving as a water absorbing agent and received in the band body; and
one or more elastic bands imparting elasticity to the band body.

2. The wrist band according to claim 1, wherein the chemical compound for the water absorbing agent is a composite in which acrylate copolymer is a primary ingredient.

3. The wrist band according to claim 1, wherein the band body is made of cloth which is porous and can absorb sweat.

4. The wrist band according to claim 1, wherein the elastic band is made of cloth having an elastic property, or of rubber.

5. The wrist band according to claim 1, wherein the cylinder-shaped band body holding the water absorbing agent therein is divided by seams extending in a longitudinal direction of an arm.

6. The wrist band according to claim 1, wherein the cylinder-shaped band body holding the water absorbing agent therein is divided by seams extending in a direction orthogonal to a longitudinal direction of an arm.

7. The wrist band according to claim 1, wherein the cylinder-shaped band body holding the water absorbing agent therein is divided by seams extending in a longitudinal direction of an arm and seams extending in a direction orthogonal to the longitudinal direction.
